(19) United States
(12) Patent Application Publication
(10) Pub. No.: US 2024/0016959 A1
(43) Pub. Date: Jan. 18, 2024

(54) RADIOPHARMACEUTICAL KETONE AND DUAL TRACER IMAGING IN ALZHEIMER'S DISEASE, COGNITIVE IMPAIRMENT, AND OTHER CONDITIONS OF ALTERED CEREBRAL METABOLISM

(71) Applicant: Cerecin Inc., Denver, CO (US)

(72) Inventors: Ernest Wong, Boulder, CO (US); Judith Walker, Edinburgh (GB); Samuel T. Henderson, Denver, CO (US)

(73) Assignee: Cerecin Inc., Denver, CO (US)

(21) Appl. No.: 18/037,681

(22) PCT Filed: Nov. 17, 2021
(86) PCT No.: PCT/US2021/059659

§ 371 Date: May 18, 2023

Related U.S. Application Data
(60) Provisional application No. 63/115,899, filed on Nov. 19, 2020.

(51) Int. Cl. A61K 51/04

(52) U.S. Cl. CPC A61K 51/0402

(57) ABSTRACT

In certain aspects, the present disclosure relates to novel radiopharmaceutical ketone molecules, including acetoacetate (AcAc) and/or beta-hydroxybutyrate (BHB), and methods of using one or more radiopharmaceutical ketone molecules or radiopharmaceutical compositions as imaging agents or tracers in positron emission tomography (PET) or combination PET and magnetic resonance imaging (MRI), e.g., as ketone-PET tracers. The methods may be used in diagnostic imaging methodologies in Alzheimer's disease, cognitive impairment, and other conditions of altered cerebral metabolism.

### RELATED APPLICATIONS

This application claims the benefit of U.S. Provisional Application No. 63/115,899, filed Nov. 19, 2020, the disclosure of which is hereby incorporated by reference in its entirety.

### FIELD OF THE INVENTION

This disclosure relates to radiopharmaceutical ketone molecules, and diagnostic imaging and tracer methodologies in Alzheimer's disease, cognitive impairment, and other conditions of altered cerebral metabolism.

### BACKGROUND OF THE INVENTION

The brain is one of the more metabolically active organs in the body and under normal conditions uses glucose as its energy resource and has very limited reserves for anaerobic metabolism. (Sokoloff L, Relation between physiological function and energy metabolism in the central nervous system, J Neurochem. 1977 July; 29(1):13-26. Review.) Glucose is the brain's main energy substrate, but in conditions of glucose deficiency, ketones (acetoacetate [AcAc] and β-hydroxybutyrate) are the main alternative energy substrates for the brain.

A prominent feature of Alzheimer's disease (AD) are regionally-specific decreases in cerebral glucose metabolism, which appear very early in the disease and may contribute to cognitive decline as well as to AD pathology. (Mosconi L, et al., Declining brain glucose metabolism in normal individuals with a maternal history of Alzheimer disease, Neurology. 2009 Feb. 10; 72(6):513-20. doi: 10.1212/01.wnl.0000333247.51383.43. Epub 2008 November 12.) Brain imaging studies have shown that defects in glucose metabolism are more prominent in AD than in the normal aging process. (Cunnane S, et al., Brain fuel metabolism, aging, and Alzheimer's disease, Nutrition. 2011 January; 27(1):3-20. doi: 10.1016/j.nut.2010.07.021. Epub 2010 October 29. Review.)

Positron emission tomography (PET) enables the minimally-invasive study of functional processes in the brain. Brain energy metabolism has been widely studied by PET using the most common PET tracer, ¹⁸F-fluorodeoxyglucose (¹⁸F-FDG), a glucose analog. Dual tracer studies using a radiolabeled ¹¹C-acetoacetate (¹¹C-AcAc) and ¹⁸F-FDG to measure brain ketone metabolism has also been used. (Roy M, et al., A Dual Tracer PET-MRI Protocol for the Quantitative Measure of Regional Brain Energy Substrates Uptake in the Rat, J of Vis. Exp. 2013 December 28:82: e50761, doi:10.3791/50761.)

While the disclosed imaging methods provide a powerful tool for comparative studies of brain metabolic pathways, there is a need in the art for improved tracers for use in such studies to provide improved assessment of cerebral activity and performance. The present invention addresses such need.

### SUMMARY OF THE INVENTION

In a first aspect, the disclosure relates to a method for diagnosing, staging, or treating a disorder in a subject. In certain embodiments, the method comprises: administering a first radiopharmaceutical composition to the subject; and acquiring a first image of an organ or tissue of interest using positron emission tomography (PET) alone or in combination with magnetic resonance imaging (MRI). In certain embodiments, the first radiopharmaceutical composition comprises a radiolabeled acetoacetate (AcAc) and/or a radiolabeled beta-hydroxybutyrate (BHB), or a prodrug, pharmaceutically acceptable salt, metabolite, solvate, hydrate, radioisotope, or combination thereof, and which is capable of being retained within the organ or tissue sufficient to emit positrons for detection by positron emission tomography.

In other embodiments, the methods further comprise: administering a second radio pharmaceutical composition to the subject; acquiring a second image of the organ or tissue of interest using PET alone or in combination with MRI; and comparing the first and second acquired images. In certain embodiments, the second radiopharmaceutical composition is administered after the first radiopharmaceutical composition is administered and the first image is acquired. In other embodiments, the compositions and images are simultaneously acquired. In certain embodiments, the second radiopharmaceutical composition comprises a radiolabeled acetoacetate (AcAc) and/or a radiolabeled beta-hydroxybutyrate (BHB), or a prodrug, pharmaceutically acceptable salt, metabolite, solvate, hydrate, radioisotope, or combination thereof, and which is capable of being retained within the organ or tissue sufficient to emit positrons for detection by positron emission tomography and which is different from the first radiopharmaceutical composition.

In certain embodiments, the methods of the disclosure further comprise determining CMRket, and CMRtot based on the first and second acquired images.

In certain embodiments, the first radiopharmaceutical composition comprises ¹⁸F-AcAc, ¹⁸F-BHB, ¹¹C-BHB, or a combination thereof. In other embodiments, the first radiopharmaceutical composition comprises ¹⁸F-AcAc and/or ¹⁸F-BHB, and the second radiopharmaceutical composition comprises ¹¹C-BHB and/or ¹⁸F-FDG.

In yet other embodiments, the methods further comprise providing a suitable medical treatment to the subject for the disorder based on the results obtained from imaging the organ or tissue of interest using positron emission tomography. In certain embodiments, the methods further comprise providing the subject with a medical treatment or management program to treat, ameliorate, or prevent the progression of the disorder.

In yet other embodiments, the methods further comprise monitoring cognitive impairment progression in a subject over time by assessing changes in ketone and/or glucose uptake over time as compared to a baseline. In some embodiments, the changes in ketone and/or glucose uptake are assessed in a medically treated state and/or untreated state.

In certain embodiments, the disorder is selected from diseases or conditions of altered cerebral metabolism. In certain embodiments, the diseases or conditions of altered cerebral metabolism are diseases or conditions of cognitive impairment or neurodegenerative disorders. In certain embodiments, the diseases or conditions of cognitive impairment or neurodegenerative disorder are selected from Age Associated Memory Impairment, Mild Cognitive Impairment, Alzheimer's Disease, Frontotemporal Dementia, Vascular and Mixed Dementias, or traumatic brain injury.

In certain embodiments, the subject is at risk for developing a disorder selected from diseases and conditions of cognitive impairment or neurodegenerative disorder. In certain embodiments, the subject's risk for developing a disease or condition of cognitive impairment or neurodegenerative disorder is determined via use of genetic markers, APOE status, or family history.

While multiple embodiments are disclosed, still other embodiments of the present disclosure will become apparent to those skilled in the art from the following detailed description, which shows and describes illustrative embodiments of the disclosure. As will be realized, the invention is capable of modifications in various aspects, all without departing from the spirit and scope of the present disclosure. Accordingly, the detailed descriptions are to be regarded as illustrative in nature and not restrictive.

### BRIEF DESCRIPTION OF THE DRAWINGS

FIG. 1 illustrates an exemplary route for synthesizing radiolabeled AcAc, in accordance with embodiments of the disclosure.

FIG. 2 illustrates an exemplary dual tracer imaging methodology, in accordance with embodiments of the disclosure.

### DETAILED DESCRIPTION OF THE INVENTION

In certain aspects, the present disclosure relates to novel radiopharmaceutical ketone molecules, including acetoacetate (AcAc) and/or beta-hydroxybutyrate (BHB), a prodrug, pharmaceutically acceptable salt, metabolite, solvate, hydrate, radioisotope, or composition thereof. The radiopharmaceutical ketone molecules may be labeled with suitable radionuclides used in imaging such as positron emission tomography (PET) or magnetic resonance imaging (MRI). By way of example, such radionuclides may be isotopes with short half-lives, such as carbon-11 (˜20 min), nitrogen-13 (˜10 min), oxygen-15 (˜2 min), fluorine-18 (˜110 min), gallium-68 (˜67 min), zirconium-89 (˜78.41 hours), or rubidium-82 (˜1.27 min).

As used herein, the term acetoacetate (AcAc) refers to a beta keto acid having the chemical formula CH₃COCH₂COCH₃. The term beta-hydroxybutyrate (BHB) refers to a beta hydroxyl acid having the chemical formula CH₃CH(OH)CH₂CO₂H. Beta-hydroxybutyric acid is a chiral compound with two enantiomers: D-β-hydroxybutyric acid and L-β-hydroxybutyric acid. In particular embodiments, novel radiopharmaceutical ketone molecules of the disclosure include ¹⁸F-AcAc and ¹⁸F-BHB, as well as ¹¹C-BHB, although any combination of AcAc and BHB with the above radionuclides are envisioned as within the scope of the disclosure.

Exemplary BIB radiopharmaceuticals of the disclosure are shown below in formulas (I) and (II). In one embodiment, the BHB radiopharmaceutical comprise (S[¹⁸F]γ-fluoro-β-hydroxybutyric acid in one embodiment, the BHB radiopharmaceutical comprises the compound of formula (I):

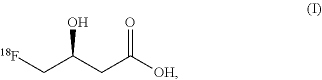

or a prodrug, pharmaceutically acceptable salt, metabolite, solvate, hydrate, or radioisotope thereof.

In another embodiment, the BHB radiopharmaceutical comprises (R)-[¹⁸F]γ-fluoro-β-hydroxybutyric acid. In one embodiment, the BHB radiopharmaceutical comprises the compound of formula (II):

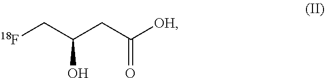

or a prodrug, pharmaceutically acceptable salt, metabolite, solvate, hydrate, or radioisotope thereof.

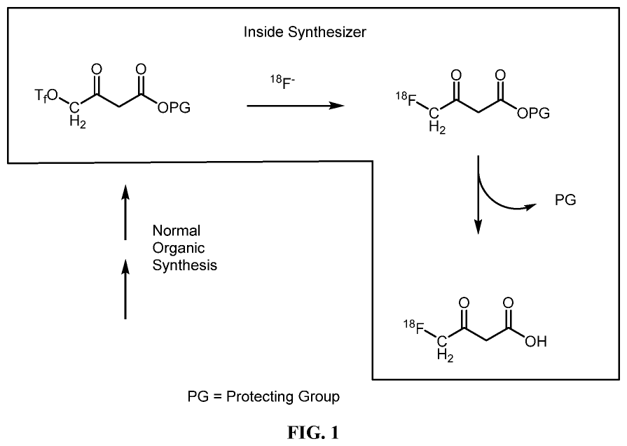

An exemplary synthesis methodology for an AcAc radiopharmaceutical in accordance with embodiments of the disclosure is provided FIG. 1.

In one aspect, the disclosure comprises pharmaceutical compositions comprising the radiopharmaceuticals described herein in combination with one or more pharmaceutically acceptable carriers. Those skilled in the art are familiar with any pharmaceutically acceptable carrier useful in the context of radiopharmaceutical compositions.

In certain embodiments, pharmaceutical compositions may be in the form of liquids and solutions suitable for intravenous injection in liquid dosage forms as appropriate and in unit dosage forms suitable for easy administration of fixed dosages. The dosage of the radiopharmaceutical depends upon many factors that are well known to those skilled in the art, for example, the type and pharmacodynamic characteristics of the radiopharmaceutical; age, weight and general health condition of the subject; nature and extent of symptoms; any concurrent therapeutic treatments; frequency of treatment and the effect desired.

In another embodiment, methods of using one or more radiopharmaceutical ketone molecules or radiopharmaceutical compositions as imaging agents or tracers in positron emission tomography (PET) or combination PET and magnetic resonance imaging (MRI) are provided, e.g., as ketone-PET tracers.

In certain embodiments, exemplary radiopharmaceutical ketone molecules are provided, including: ¹¹C-BHB, ¹⁸F-AcAc, and/or ¹⁸F-BHB, etc. In other embodiments, radiopharmaceutical compositions are provided comprising combinations of a radiopharmaceutical glucose substrate, e.g., a fluorinated-fluorodeoxyglucose compound (FDG) such as ¹⁸F-fluorodeoxyglucose (¹⁸F-FDG) and one or more radiopharmaceutical ketone molecules of the disclosure (e.g., ¹¹C-BHB, ¹⁸F-AcAc, and/or ¹⁸F-BHB) are provided.

In certain embodiments, diagnostic imaging methods using the radiopharmaceutical ketone molecules and radiopharmaceutical compositions of the disclosure are provided. In other embodiments, dual tracer imaging methods utilizing radiopharmaceutical compositions comprising a combination of a glucose substrate, e.g., a fluorinated-fluorodeoxyglucose compound (FDG) such as ¹⁸F-fluorodeoxyglucose (¹⁸F-FDG), and one or more radiopharmaceutical ketone molecules of the disclosure are provided.

In one embodiment, the disclosure comprises a method for diagnosing, staging or treating a disorder in a subject comprising: administering a radiopharmaceutical composition to the subject; and imaging an organ or tissue of interest using positron emission tomography (PET) alone or in combination with magnetic resonance imaging (MRI); wherein the radiopharmaceutical composition comprises a radiolabeled AcAc, a radiolabeled BHB, a prodrug, pharmaceutically acceptable salt, metabolite, solvate, hydrate, radioisotope, or composition thereof, and is capable of being retained within the organ or tissue sufficient to emit positrons for detection by positron emission tomography.

In one embodiment, the method further comprises providing a suitable medical treatment to the subject for the disorder based on the results obtained from imaging the organ or tissue of interest using positron emission tomography. The subject may then be provided with a medical treatment or management program to treat, ameliorate, or prevent the progression of the disorder.

As used herein, the term “medical treatment” or “management program” refers to an effective treatment modality or program to include pharmacologic and non-pharmacologic components for treating, ameliorating, and/or preventing the disorder. As used herein, the terms “treatment,” “treating,” “treat,” and the like, refer to obtaining a desired pharmacologic and/or physiologic effect. The effect can be prophylactic in terms of completely or partially preventing the disorder or symptoms thereof and/or can be therapeutic in terms of a partial or complete cure for the disorder and/or adverse effect attributable to the disorder. “Treatment” covers any treatment of a disorder in a subject, particularly in a human, and includes: (a) preventing the disorder in a subject which may be predisposed to the disorder but has not yet been diagnosed as having it; (b) inhibiting the disorder, i.e., arresting its development; and (c) relieving the disorder, i.e., causing regression of the disorder and/or relieving one or more symptoms of the disorder. “Treatment” can also encompass delivery of an agent or administration of a therapy in order to provide for a pharmacologic effect.

In one aspect, the invention comprises a method for monitoring (e.g., detecting positive metabolic changes in response to treatment) a disorder in a subject comprising: administering a radiopharmaceutical composition to a subject undergoing medical treatment for the disorder; imaging an organ or tissue of interest using positron emission tomography; and comparing the quantity or distribution of the radiopharmaceutical present in the subject with a control quantity or distribution indicative of the effectiveness of the medical treatment; wherein the radiopharmaceutical composition comprises a radiolabeled AcAc, a radiolabeled BHB, a prodrug, pharmaceutically acceptable salt, metabolite, solvate, hydrate, radioisotope, or composition thereof, and is capable of being retained within the organ or tissue sufficient to emit positrons for detection by positron emission tomography.

Without being limited, imagining methods similar to those disclosed by Roy M, et al., A Dual Tracer PET-MRI Protocol for the Quantitative Measure of Regional Brain Energy Substrates Uptake in the Rat. J. of Vis. Exp. 2013 December 28:82: e50761, doi:10.3791/50761 (the contents of which is herein incorporated by reference in its entirety) may be used in connection with the imaging methods of the disclosure, modified as may be understood by those of skill in the art to utilize the disclosed tracer compounds and subject populations.

In accordance with embodiments of the disclosure, the methods may be used to, e.g., determine CMRket, and CMRtot (CMRket plus CMRglu), select subjects for treatment with interventions designed to optimize cerebral metabolism, determine response to treatment with interventions designed to optimize cerebral metabolism.

For instance, without being limited by theory, exemplary calculations may be performed as follows:

$$\begin{matrix}\frac{{C}_{Tissue} ( t )}{{C}_{P} ( t )} = K \frac{{\int }_{0}^{t} ( {C}_{P} ( t ) dt )}{{C}_{P} ( t )} + V & ( 1 ) \\ {CMR}_{glc} = \frac{K * [ glucose ]}{LC} & ( 2 ) \\ {CMR}_{AcAc} = K * [ AcAc ] & ( 3 )\end{matrix}$$

This model uses equation 1, where the measured PET activity (C_{Tissue}) is divided by plasma activity (C_{P}) and plotted at a normalized time. The variable K represents brain influx and V is the tracers distribution volume. Cerebral metabolic rate of glucose (CMR_{glc}) and AcAc/ketone (CMR_{AcAc}) are calculated according to equations 2 and 3. Brain influx and plasma concentrations are required. For CMR_{glc}, the lumped constant (LC) is used to convert the radiolabeled glucose substrate (¹⁸F-FDG) uptake to glucose uptake. An LC value of 0.48 has been reported in the literature for rats. No constant is used for CMR_{AcAc} calculation as the radiotracer has the exact same chemical formula as the endogenous molecule.

In certain embodiments, the methods may be used in subjects with conditions or disorder of altered cerebral metabolism, e.g., with cognitive impairment (whether due to Age Associated Memory Impairment, Mild Cognitive Impairment, Alzheimer's Disease, Frontotemporal Dementia, Vascular and Mixed Dementias, traumatic brain injury, and other causes of cognitive impairment), at risk for cognitive impairment (as determined by any means, such as genetic markers, APOE status, family history, etc.), at risk for other neurodegenerative disorders, etc.

In certain embodiments, the imaging methods may be used to monitor cognitive impairment progression in a subject over time, e.g., by assessing changes in ketone uptake over time, e.g., as compared to a baseline. Alternatively, changes in glucose as compared to ketone uptake over time may be monitored to assess cognitive impairment progression in a subject over time. In other embodiments, the changes in ketone uptake or glucose compared to ketone uptake may be assessed in a treated and/or untreated state.

In yet other embodiments the imaging methods may be used to assess the effectiveness of a cognitive impairment treatment regimen in a subject by, e.g., monitoring the changes in ketone uptake, e.g., as compared to a baseline. Alternatively, changes in glucose vs. ketone uptake may be monitored to assess effectiveness of a neurodegenerative disease treatment regimen.

As used herein, the term “subject” means a human or other mammalian subject. Non-human subjects may include primates, livestock animals (e.g., sheep, cows, horses, goats, pigs) domestic companion animals (e.g., cats, dogs) laboratory test animals (e.g., mice, rats, guinea pigs, rabbits) or captive wild animals.

### Examples

The following examples are provided for illustrative purposes only and are not intended to limit the scope of the invention.

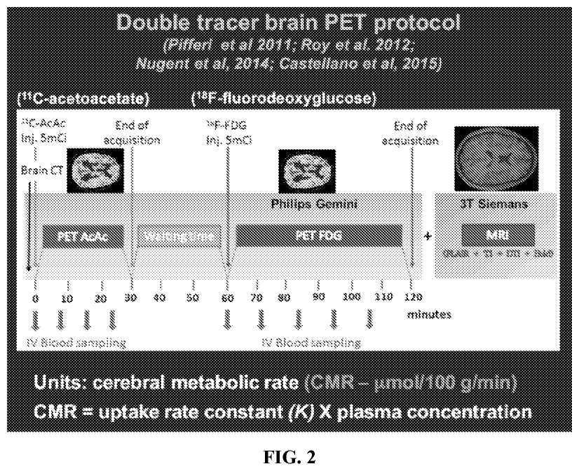

Again, as illustrated in FIG. 2 and generally disclosed in Roy M, et al., ¹¹C-AcAc in combination with ¹⁸F-FDG has been used in a dual tracer brain PET. A similar protocol may be used in connection with the present imaging methods. In this regard, in accordance with the present disclosure, ¹⁸F-AcAc and/or ¹⁸F-BHB may be used alone or in combination with ¹¹C-BHB and/or ¹⁸F-FDG in a similar manner to provide similar images.

All publications and patent applications cited in this specification are herein incorporated by reference as if each individual publication or patent application were specifically, and individually, indicated to be incorporated by reference.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A method for diagnosing, staging, or treating a disorder in a subject comprising:
administering a first radiopharmaceutical composition to the subject; and
acquiring a first image of an organ or tissue of interest using positron emission tomography (PET) alone or in combination with magnetic resonance imaging (MRI);
wherein the first radiopharmaceutical composition comprises a radiolabeled acetoacetate (AcAc) and/or a radiolabeled beta-hydroxybutyrate (BHB), or a prodrug, pharmaceutically acceptable salt, metabolite, solvate, hydrate, radioisotope, or combination thereof, and which is capable of being retained within the organ or tissue sufficient to emit positrons for detection by positron emission tomography.

2. The method of claim 1, further comprising:
administering a second radio pharmaceutical composition to the subject;
acquiring a second image of the organ or tissue of interest using PET alone or in combination with MRI; and
comparing the first and second acquired images;
wherein the second radiopharmaceutical composition comprises a radiolabeled acetoacetate (AcAc) and/or a radiolabeled beta-hydroxybutyrate (BHB), or a prodrug, pharmaceutically acceptable salt, metabolite, solvate, hydrate, radioisotope, or combination thereof, and which is capable of being retained within the organ or tissue sufficient to emit positrons for detection by positron emission tomography and which is different from the first radiopharmaceutical composition.

3. The method of claim 2, further comprising determining CMRket, and CMRtot based on the first and second acquired images.

4. The method of claim 1, further comprising providing a suitable medical treatment to the subject for the disorder based on the results obtained from imaging the organ or tissue of interest using positron emission tomography.

5. The method of claim 4, further comprising providing the subject with a medical treatment or management program to treat, ameliorate, or prevent the progression of the disorder.

6. The method of claim 1, further comprising monitoring cognitive impairment progression in a subject over time by assessing changes in ketone and/or glucose uptake over time as compared to a baseline.

7. The method of claim 6, wherein the changes in ketone and/or glucose uptake are assessed in a medically treated state.

8. The method of claim 1, wherein the disorder is selected from diseases or conditions of altered cerebral metabolism.

9. The method of claim 8, wherein the diseases or conditions of altered cerebral metabolism are cognitive impairment or neurodegenerative disorders.

10. The method of claim 9, wherein the diseases or conditions of cognitive impairment or neurodegenerative disorders are selected from Age Associated Memory Impairment, Mild Cognitive Impairment, Alzheimer's Disease, Frontotemporal Dementia, Vascular and Mixed Dementias, or traumatic brain injury.

11. The method of claim 1, wherein the subject is at risk for developing a disorder selected from diseases and conditions of cognitive impairment or neurodegenerative disorder.

12. The method of claim 11, wherein the subject's risk for developing a disease or condition of cognitive impairment or neurodegenerative disorder is determined via use of genetic markers, APOE status, or family history.

13. The method of claim 1, wherein the first radiopharmaceutical composition comprises ¹⁸F-AcAc, ¹⁸F-BHB, ¹¹C-BHB, or a combination thereof.

14. The method of claim 2, wherein the first radiopharmaceutical composition comprises ¹⁸F-AcAc and/or ¹⁸F-BHB, and the second radiopharmaceutical composition comprises ¹¹C-BHB and/or ¹⁸F-FDG.
